(19) United States
(12) Patent Application Publication
(10) Pub. No.: US 2024/0197417 A1
(43) Pub. Date: Jun. 20, 2024

(54) SURGICAL INSTRUMENT

(71) Applicant: KARL STORZ SE & CO. KG, Tuttlingen (DE)

(72) Inventors: Sven Axel Grüner, Tuttlingen (DE); Jochen Stefan, Tuttlingen (DE)

(73) Assignee: KARL STORZ SE & CO. KG, Tuttlingen (DE)

(21) Appl. No.: 18/536,554

(22) Filed: Dec. 12, 2023

(30) Foreign Application Priority Data
Dec. 20, 2022 (DE) 102022134205.0

(51) Int. Cl. A61B 34/30; A61B 34/00; A61B 46/10

(52) U.S. Cl. CPC A61B 34/30; A61B 34/71; A61B 46/10

(57) ABSTRACT

The present disclosure relates to a surgical instrument having a housing arranged at the proximal end of the surgical instrument, an interface arranged on the housing for coupling to a drive unit, a steering gear arranged in the housing, wherein the steering gear has a spatially orientable swash plate, and at least one spindle drive for driving the steering gear. The present disclosure further relates to a sterile drive system for such a surgical instrument and to a method for sterile mounting of a drive system.

### CROSS-REFERENCE TO RELATED APPLICATIONS

This application claims priority of German Patent Application No. 10 2022 134 205.0 filed on Dec. 20, 2022, the contents of which are incorporated herein.

### TECHNICAL FIELD

The present disclosure relates to a surgical instrument. Furthermore, the present disclosure relates to a sterile drive system for such a surgical instrument. The present disclosure also relates to a method for mounting such a sterile drive system.

### BACKGROUND

From the prior art, surgical instruments are known which can be guided manually or by a robot and which have tools whose tool tip can be pivoted by means of a plurality of pivoting members engaging in one another. These pivoting members are connected to a plurality of steering wires or steering cables in order to achieve sensitive control of the tool tip. The steering wires can be used to obtain a uniform distribution of forces in all unwinding directions.

U.S. Pat. No. 10,485,621 B2 discloses a drive unit which can be attached to a robot and which has an interface surface for coupling to a surgical instrument. Various mechanical and electrical interfaces are arranged on the interface surface for the purpose of transmitting mechanical movements and electrical signals. U.S. Pat. No. 10,485,621 B2 further discloses a covering for a robot arm, which has an opening for the drive unit and the surgical instrument that can be coupled to the drive unit.

A surgical instrument having a steering gear is known from DE 10 2019 121 092 A1. The steering gear allows the positioning angles of two drives to be transferred directly to a spatially adjustable plate (swash plate) in order to align the latter to control the tip of the tool. To do this, steering wires are fastened to the swash plate so that the tip of the tool can be steplessly and smoothly controlled by alignment of the swash plate. U.S. Pat. No. 10,105,128 B2 also discloses a gear system for a surgical instrument. The gear system of this surgical instrument comprises a displaceable disk and articulated rods arranged for moving the displaceable disk.

### SUMMARY

Proceeding from this prior art, it is an object of the present disclosure to make available a surgical instrument which can be coupled to a drive unit, attachable to a robot arm, in such a way that the surgical instrument remains sterile in an intraoperative change.

This object is achieved with a surgical instrument according to claim 1. Further embodiments are set forth in the dependent claims.

The surgical instrument according to the disclosure comprises a housing arranged at the proximal end of the surgical instrument, an interface arranged on the housing for coupling to a drive unit, a steering gear arranged in the housing, wherein the steering gear has a spatially orientable swash plate, and at least one spindle drive for driving the steering gear.

The steering gear having a swash plate can provide a surgical instrument which can be coupled proximally to a robot-side drive unit and which enables a sterile intraoperative change of the surgical instruments.

The interface can be arranged on a coupling surface of the housing extending perpendicular to the longitudinal axis of a shaft, the shaft passing through the coupling surface. In other words, the shaft extends through the coupling surface and runs perpendicularly to this coupling surface. Starting from the housing arranged at the proximal end of the surgical instrument, the shaft extends in the direction of the distal end of the surgical instrument, where the actual tool of the surgical instrument is arranged.

The interface can comprise drive plates which drive the steering gear to pivot the swash plate and which can thus pivot the pivoting members mentioned at the outset. The drive plates can be arranged on the coupling surface. The drive plates can be coupled to the motor drive plates on the robot-side drive unit. At least on the motor drive plates, a sterile adapter plate can be provided in order to cover the non-sterile drives. The sterile adapter plate can have rotatable elements which allow a torque-transmitting coupling between the drive plates at the interface of the surgical instrument and the motor drive plates.

The surgical instrument can have at least a first spindle drive and a second spindle drive. The axes of rotation of the first spindle drive and of the second spindle drive can be parallel to each other and to the longitudinal axis of the surgical instrument. The surgical instrument can be coupled to the drive unit in the direction of its longitudinal axis. This means that the coupling direction for coupling the surgical instrument to the drive unit is parallel to the longitudinal axis and thus parallel to the axes of rotation of the spindle drives.

The first spindle drive and the second spindle drive can each have a drive plate. The drive plates can be arranged on the coupling surface. In other words, the drive plates are arranged at the interface.

The steering gear can have a first slide and a second slide. The first slide can be assigned to the first spindle drive and the second slide to the second spindle drive. The first slide and the second slide can be moved along the axis of rotation of the respective spindle drive via the spindle drives assigned to them. The first slide and the second slide can be moved relative to each other along the longitudinal axis of the surgical instrument via the spindle drives assigned to them. In other words, the slides can change their relative positions relative to each other.

The steering gear can have a clamp. The clamp can be arranged on the swash plate. The clamp can be coupled to the first slide and to the second slide. Through changes in their relative positions to each other, the first slide and the second slide can move the clamp and thereby drive the swash plate to perform certain movement patterns.

The first slide and the second slide can each have a ball head. The clamp can have at least two ball sockets. The ball heads on the slides can each engage in one of the ball sockets on the clamp and thereby couple the clamp to the slides. Through the change in the relative positions in the axial direction between the first slide and the second slide, the distance between the ball heads on the first slide and the second slide can change. Through the changes in the distances between the ball head on the first slide and the ball head on the second slide, the clamp can be moved. The ball heads of the slides and the ball sockets on the clamp can provide an articulated coupling of the clamp to the slides, so that the clamp can perform articulated movements.

The clamp can have a clamp base and two clamp arms. The clamp arms can each have an end portion formed by a free end of the clamp arms. Each of the ball sockets can be arranged respectively on an end portion of a clamp arm. The ball sockets on the end portions of the clamp arms can be coupled to the ball heads on the slides.

The clamp arms can define a clamp opening between their end portions. The clamp opening can lie opposite the clamp base. The clamp can be brought into engagement with the swash plate via the clamp opening. By connecting the clamp to the swash plate, the movement patterns generated by the slides can be transferred from the clamp to the swash plate and thus to the pivoting members of the surgical instrument tool.

The clamp can be elastic at least in some sections. This allows the clamp to elastically absorb deformations which arise from the axial movements of the first slide and of the second slide and from the resulting change in distances between the ball heads. This also permits the use of stronger materials that can absorb the high pressures on the ball heads.

The elasticity of the clamp can be adjusted at least via the geometric thickness and/or the material used for the clamp. In this case, the expression “geometric thickness” can refer to the radial thickness of the clamp. If a portion of the clamp is made thinner or slimmer, the clamp can take up the above-described deformations without the clamp being plastically and thus permanently deformed.

The clamp can have axial contact surfaces for the swash plate. With its axial contact surfaces, the clamp can rest at least in part on the swash plate or support itself on the swash plate. The axial contact surfaces of the clamp can extend parallel to each other. The axial contact surfaces of the swash plate are arranged in opposite directions to each other on the clamp. In other words, the axial contact surfaces of the clamp can point away from each other.

The swash plate can have a groove. The groove can extend all around the swash plate. The groove can have axial contact surfaces for the clamp. The axial contact surfaces of the groove can extend substantially parallel to each other. The axial contact surfaces of the groove can be formed opposite each other. The clamp can be received in the groove. Once the clamp is received in the groove, the axial contact surfaces of the clamp and the axial contact surfaces of the groove can bear on each other.

The clamp can enclose the swash plate at least partially. The clamp can enclose the swash plate almost completely. The clamp can surround more than 180°, in particular an angular range of between 270° and 350° of the circumference of the swash plate.

Since the clamp can surround the swash plate almost completely, a circumferential contact surface can be provided between the clamp and the swash plate. This relatively large circumferential contact surface can be made available by the contact between the inner surface of the clamp and the groove bottom of the groove of the swash plate. The inner side of the clamp can contact the groove bottom of the groove of the swash plate. The circumferential contact surface between the clamp and the swash plate enables improved guidance of the transfer of the movements from the swash plate and prevents tilting or jamming of the swash plate.

The steering gear can have a steering ring coupled to the swash plate. The steering ring can surround the swash plate on the outer circumference thereof.

The first spindle drive and the second spindle drive can each interact with a gearwheel for driving at least two bevel gears. To move the steering ring, the bevel gears can be coupled thereto in a torque-transmitting manner. The bevel gears can each be mounted on the housing via a bearing shaft so as to be rotatable about an axis of rotation. The bevel gears can have an all-round or complete bevel toothing. The gearwheels interacting with the spindle drives can be mounted rotatably on the bearing shafts of the bevel gears.

The steering ring can have at least two bevel toothing portions. The bevel toothing portions can be segmented. This means that the bevel toothing portions can be configured in the form of segments of a circle. For example, the bevel toothing portions can extend over an angular extent of 60° to 120°. The bevel toothing portions can engage with the bevel gears so as to be able to move the steering ring.

The bevel toothing portions can be formed in one piece with the steering ring. The bevel toothing portions can also be formed independently of the steering ring as a separate component or as separate components. In this case, the bevel toothing portions can be connected to the steering ring in a torque-transmitting manner. With a two-part or multi-part design of the steering ring and of the bevel toothing portions to be connected thereto, production of these components can be kept simple.

The gearwheels allow the bevel gears to be moved in order to move the steering ring and the swash plate coupled thereto. If the bevel gears are driven accordingly, the steering ring can be pivoted about the axis of rotation formed by the bearing shafts or can be rotated about an axis of rotation perpendicular to this axis of rotation.

The present disclosure further relates to a drive system for a surgical instrument. The drive system comprises a drive unit which can be attached to a robot arm, a sterile adapter plate which is attached to the drive unit and at least partially covers an interface on the drive unit, and the surgical instrument of the type described above, wherein the surgical instrument is coupled to the drive unit from the proximal direction via the interface.

The sterile adapter plate can be designed such that it has rotatable clutch disks, which provide a torque-transmitting coupling between the drive plates at the interface of the surgical instrument and the non-sterile motor drive plates of the drive unit. The rotatable clutch disks can permit transfer of the rotation from the non-sterile motors of the drive unit to the sterile surgical instrument and in particular to the gear system of the surgical instrument.

The sterile adapter plate can be part of a sterile adapter that at least partially surrounds the drive unit. Such a sterile adapter can be plugged with the sterile adapter plate onto the drive unit.

Furthermore, at least the sterile adapter plate can be connected to a sterile covering which surrounds at least a part of the drive unit. For example, the sterile covering can be formed by a film.

The present disclosure further relates to a method for sterile mounting of a drive system for a surgical instrument, wherein the drive system comprises a drive unit, which can be attached to a robot arm, and the surgical instrument described above, wherein the method comprises the following steps:
attaching a sterile adapter plate to the drive unit, which at least partially covers the interface on the drive unit, and
attaching the surgical instrument to the interface of the drive unit from the proximal direction.

In connection with the method for sterile mounting of the drive system, it should also be mentioned that the sterile adapter plate can be connected to a sterile covering which at least partially surrounds the drive unit. The sterile covering can also partially surround a guide device, via which the drive unit can be connected to a robot arm.

Attaching the sterile adapter plate to the drive unit can be the first step. The sterile adapter plate covers the non-sterile motor drive plates of the drive unit and has rotatable clutch disks that allow rotation to be transmitted from the motor drive plates to the drive plates of the surgical instrument.

In the second step, the surgical instrument, with the steering gear comprising a swash plate, is plugged from the proximal direction onto the sterile adapter plate and thus onto the drive unit. This coupling of the surgical instrument to the drive unit can take place in the direction of the longitudinal axis of the surgical instrument. This joining direction is on a line with a trocar, so that the surgical instrument, in an operation, could then be pushed in a continuous movement into the patient.

The disclosure is explained below by way of example and with reference to figures. The drawing, the description and the claims contain numerous features in combination. A person skilled in the art will advantageously also consider the features individually and use them in combination within the scope of the claims.

If there is more than one instance of a given object, only one of them may be referenced in the figures and in the description. The description of this instance can be transferred accordingly to the other instances of the object. If objects are named in particular by means of numerical words, such as first, second, third object, etc., these are used to name and/or assign objects. Thus, for example, a first object and a third object can be included, but no second object. However, a number and/or a sequence of objects could also be derived from numerical words. The drawings, the description and the claims contain numerous features in combination. It will be appreciated that the features mentioned above and the features yet to be explained below can be applied not only in the respectively specified combination but also in other combinations or on their own, without departing from the scope of the present disclosure.

### BRIEF DESCRIPTION OF DRAWINGS

FIG. 1 shows a partially cut-away perspective view of a surgical instrument, according to a first embodiment, and a drive unit in the coupled state;

FIG. 2 shows a partially cut-away perspective view of the steering gear of the surgical instrument according to a first embodiment;

FIGS. 3 to 5 show different views of a clamp for coupling to a swash plate;

FIG. 6 shows a perspective view of a surgical instrument according to a second embodiment; and

FIGS. 7 and 8 show perspective views of a drive system.

### DETAILED DESCRIPTION OF PREFERRED EMBODIMENTS

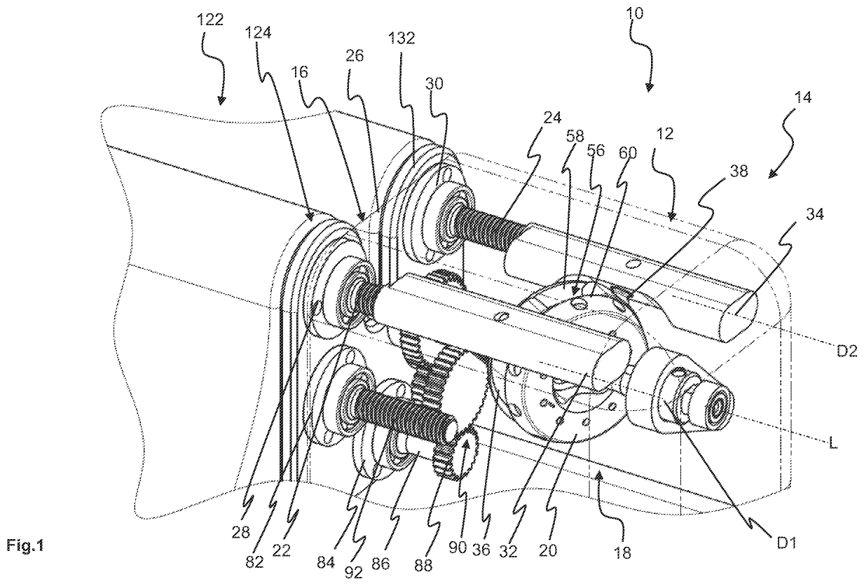

FIG. 1 shows a partially cut-away perspective view of a surgical instrument 10, according to a first embodiment, and of a drive unit 122 in the coupled state.

The surgical instrument 10 comprises a housing 12. On the housing 12, an interface 16 is formed which serves for coupling to the drive unit 122. The interface 16 is arranged on a coupling surface 26 of the housing 12 extending perpendicular to the longitudinal axis L of the surgical instrument 10. The longitudinal axis L runs through the coupling surface 26.

The interface 16 comprises drive plates 28 and 30, which are arranged on the coupling surface 26. The drive plates 28, 30 are coupled to the first spindle drive 22 and the second spindle drive 24, respectively. The axes of rotation D1 and D2 of the first spindle drive and of the second spindle drive run parallel to each other. In addition, the axes of rotation D1 and D2 are parallel to the longitudinal axis L.

The surgical instrument 10 further comprises a steering gear 18. The steering gear comprises a first slide 32 and a second slide 34. The first slide 32 is assigned to the first spindle drive 22 and the second slide 34 to the second spindle drive 24. The spindle drives 22 and 24 can move the slides 32 and 34 along the axes of rotation D1 and D2 and thus also parallel to the longitudinal axis L. Each of the slides 32 and 34 has a ball head 38. The slides 32 and 34 are coupled to a clamp 36 via the ball heads 38. The clamp 36 serves to transfer movements to a swash plate 20 of the steering gear 18.

The clamp 36 has two ball sockets 40 and 42, which are each assigned to one of the ball heads 38. In other words, one of the ball sockets 40, 42 in each case receives a ball head 38 on one of the slides 32, 34. The ball heads 38 and the ball sockets 40, 42 couple the clamp 36 to the slides 32 and 34 in an articulated manner, such that the clamp 36 can execute articulated movements.

The slides 32 and 34 can be moved relative to each other, via the drive spindles of the spindle drives 22 and 24, along the longitudinal axis L or along the axes of rotation D1 and D2. Through the changes in the relative positions of the slides 32 and 34 relative to each other, the distances between the ball heads 38 of the slides 32 and 34 can change. The clamp 36 can be moved by the changes in the relative positions of the slides 32 and 34 and by the resulting changes in the distances between the ball heads 38 of the slides 32 and 34. The movements of the clamp 36 can be transferred to the swash plate 20, which is coupled to the clamp 36.

According to FIG. 1, the clamp 36 is received in a groove 56 of the swash plate 20. The clamp 36 largely surrounds the swash plate 20 in the region of the groove 56. In other words, the clamp 36 extends over a large part of the circumference of the groove bottom (not shown in FIG. 1) of the groove 56 of the swash plate 20 and can contact the groove bottom at least in sections.

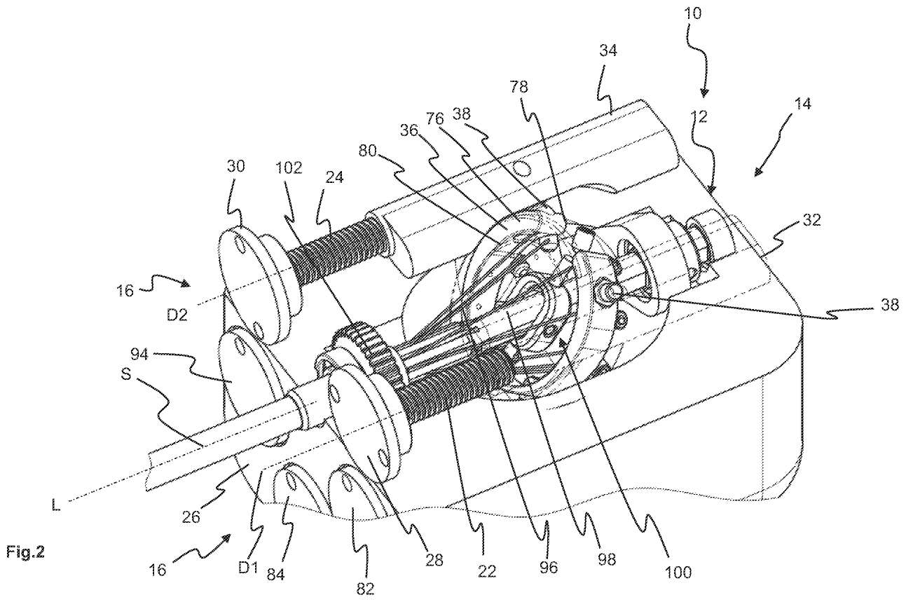

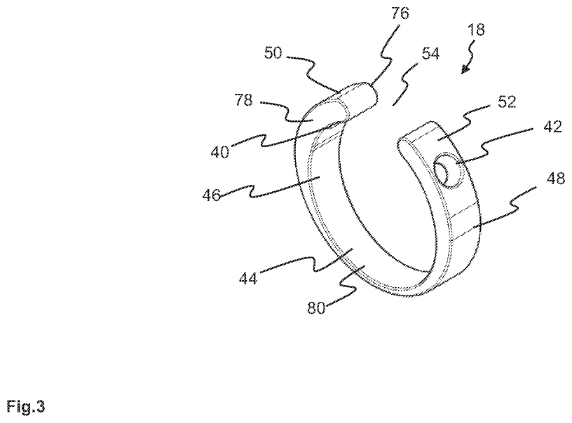

The groove 56 has axial contact surfaces 58 and 60 for the swash plate 20. The axial contact surfaces 58 and 60 lie opposite each other and extend parallel to each other. The clamp 36 also has axial contact surfaces 76 and 78, which are shown in FIG. 2. The contact surfaces 76 and 78 of the clamp 36 are formed in opposite directions to each other. The contact surfaces 76 and 78 extend substantially parallel to each other. The clamp 36 can support itself with its axial contact surfaces 76 and 78 on the axial contact surfaces 58 and 60 of the groove 56 of the swash plate 20. By the contact surfaces 58 and 60 of the groove 56 of the swash plate 20 bearing on the contact surfaces 76 and 78 of the clamp 36, jamming or tilting of the swash plate 20 can be prevented during the transfer of movements to the swash plate 20. Jamming or tilting of the swash plate 20 is also prevented by the fact that the clamp 36 largely surrounds the groove bottom of the groove 56 of the swash plate 20. The clamp 36 has an inner surface 80 (see FIG. 3), which can rest against the groove bottom. The inner surface 80 thus forms a relatively large circumferential contact surface. By virtue of the large-area contact between the clamp 36 and the groove bottom of the groove 56 of the swash plate 20, the guiding of the swash plate 20 by the clamp 36 can be improved, and tilting or jamming of the swash plate 20 can almost be ruled out.

The interface 16 comprises further drive plates 82 and 84, which are provided for driving further elements of the steering gear. For example, the drive plate 84 can drive a gear transmission 90 via a shaft 86 and a spur gear 88. For example, the gear transmission 90 can serve to move the shaft S in rotation. The drive plate 82 is connected to a further drive spindle 92, which can drive various additional functions of the surgical instrument.

FIG. 1 furthermore shows the drive unit 122, which has an interface 124. The drive unit 122 is coupled to the interface 16 of the surgical instrument 10 via its interface 124. Between the interface 124 of the drive unit 104 and the interface 16 of the surgical instrument 10, a sterile adapter plate 132 is provided.

The sterile adapter plate 132 covers the non-sterile interface 124 of the drive unit 122 and in particular the non-sterile motor drive plates of the drive unit 122. The sterile adapter plate 132 is designed to transfer the rotation of the motor drive plates (not shown) to the drive plates 28, 30, 82 and 84 at the interface 16 of the surgical instrument 10. For this purpose, the sterile adapter plate 132 has rotatable clutch disks (not shown), which allow the described transfer of rotation.

The sterile adapter plate 132 can be connected releasably to the drive unit 122. The sterile adapter plate 132 can, for example, be attached to the drive unit 122 via a click or snap connection. When the sterile adapter plate 132 is attached to the drive unit 122, the surgical instrument 10 can be connected to the drive unit 122 from the proximal direction via the sterile adapter plate 132.

FIG. 2 shows a partially cut-away perspective view of surgical instrument 10. On the coupling surface 26 of the interface 16 formed on the housing 12, a further drive plate 94 is provided in addition to the drive plates 28, 30, 82 and 84.

The drive plates 28 and 30 are connected to the spindle drives 22 and 24, which in turn drive the slides 32 and 34. The slides 32 and 34 each have a ball head 38, which each couple the slides 32, 34 to the clamp 36. Through changes in the axial relative positions of the slides 32 and 34 relative to each other, the distances between the ball heads 38 on the slides 32 and 34 can change, as a result of which the clamp 36 is moved. The movements of the clamp 36 are transferred to the swash plate 20. Steering wires 96, attached to the swash plate 20, transfer the movements of the swash plate 20 to the pivoting members of the tool on the surgical instrument 10 or the tool tip and enable sensitive control of the tool.

The steering gear 18 has a shaft 98. The swash plate 20 is connected to the shaft 98 via a cardan joint 100. The cardan joint 100 is arranged radially within the swash plate 20 and thus also radially within the clamp 36. Since the above-described circumferential contact surface between the inner face 80 of the clamp 36 and the groove bottom of the groove 56 of the swash plate 20 encloses the cardan joint 100 and in particular its center, the above-described improved guidance is achieved and tilting or jamming of the swash plate 20 is prevented. The clamp 36 serves for surface distribution of the steering forces for steering the pivoting members or the tool tip of the tool on the surgical instrument 10. On the shaft 98, a gearwheel 102 is further arranged, via which the shaft S can be moved in a rotation.

FIG. 3 shows a perspective view of the clamp 36. The clamp 36 has a clamp base 44 and two clamp arms 46 and 48. The clamp arms 46 and 48 extend from the clamp base 44. The clamp 36 also has ball sockets 40, 42. The ball sockets 40, 42 serve to accommodate the ball heads 38 arranged on the slides 32 and 34 (see FIGS. 1 and 2). The ball sockets 40, 42 are formed on the clamp arms 46 and 48. The ball sockets 40, 42 are formed at the end portions 50, 52 in the region of the free ends of the clamp arms 46 and 48. The ball sockets 46 and 48 are designed as continuous openings in the clamp 36. The clamp arms 46 and 48 define the clamp opening 54 between their end portions 50, 52. The swash plate 20 can be inserted into the clamp 36 via the clamp opening 54. The clamp opening 54 lies substantially opposite the clamp base 44. The clamp 36 has the axial contact surfaces 78 and 80 already described above, which are formed opposite to each other on the clamp 36. The contact surfaces 78 and 80 of the clamp 36 extend parallel to each other.

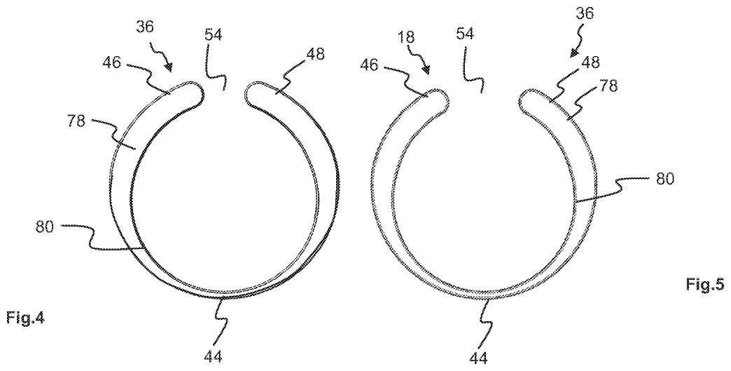

FIGS. 4 and 5 show two exemplary embodiments of the clamp 36. In FIG. 4, the clamp opening 54 is smaller compared to the clamp opening 54 according to FIG. 5. This means that the distance between the free ends of the clamp arms 46 and 48 in FIG. 4 is smaller than in FIG. 5. Through the selection of the material for the clamp 36 and the geometric thickness of the clamp 36 in the radial direction, the elastic properties of the clamp 36 can be adjusted. If, for example, the clamp 36 or at least a portion of the clamp 36 is made slimmer, or thinner in the radial direction, the clamp 36 can take up the deformation on account of the changes in the distance between the ball heads 38 on the slides 32, 34, without being plastically deformed.

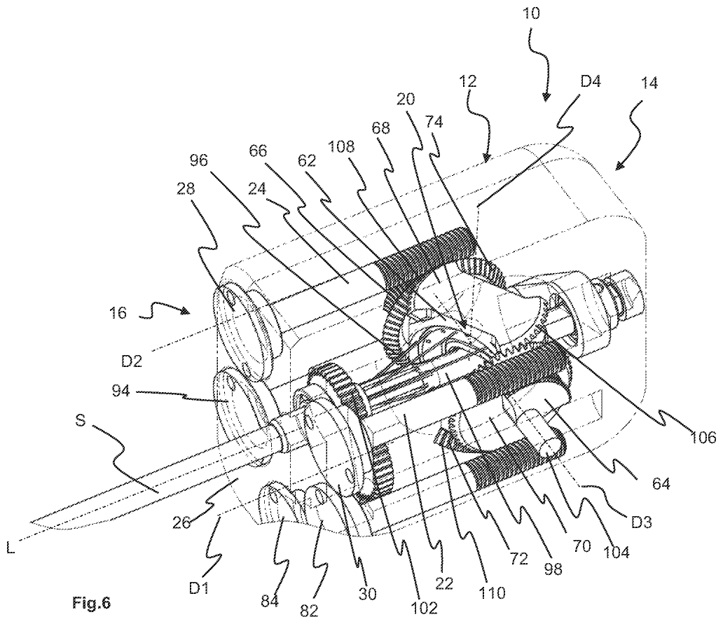

FIG. 6 shows a perspective view of a surgical instrument 10 according to a second embodiment. The surgical instrument 10 comprises the housing 12 and the interface 16. The interface 16 comprises a coupling surface 26 on which the drive plates 28, 30, 82, 84 and 94 are arranged. The coupling surface 26 extends perpendicular to the longitudinal axis L of the surgical instrument 10. Furthermore, the coupling surface 26 also extends perpendicular to the axes of rotation D1 and D2 of the first spindle drive 22 and of the second spindle drive 24. The axes of rotation D1 and D2 of the first spindle drive 22 and of the second spindle drive 24 extend parallel to each other and parallel to the longitudinal axis L of the surgical instrument 10.

The surgical instrument 10 can comprise a steering ring 62. The steering ring 62 is coupled to the swash plate 20. The swash plate 20 is accommodated inside the steering ring 62. The steering ring 62 can be driven via the spindle drives 22 and 24 to carry out movements. The spindle drives 22 and 24 each drive a gearwheel 64, 66. The gearwheels 64, 66 are each mounted on the housing 12 via a bearing shaft 104. The bearing shafts 104 define a third axis of rotation D3, about which the steering ring 18 can be rotated. The third axis of rotation D3 extends orthogonally to the axes of rotation D1, D2 and to the longitudinal axis L of the surgical instrument 10.

The gearwheels 64 and 66 are coupled to bevel gears 66 and 70. The bevel gears 66 and 70 have a bevel toothing all about the circumference. The steering ring 62 is coupled to two bevel gears 68 and 72, but these only have portions 106, 108, 112 with a bevel toothing. In other words, the bevel gears 68 and 72 are not designed as complete bevel gears, but are provided only with the bevel toothing portions 106, 108, 112. The second bevel toothing portion on the bevel gear 72 is not shown in FIG. 6. The bevel toothing portions 106 and 110 engage with the bevel gear 70. The bevel toothing portion 108 and the bevel toothing portion not shown engage with the bevel gear 66.

The steering ring 62 and bevel gears 68 and 72 are mounted on the gear housing 12 such that they can be rotated about a fourth axis of rotation D4. The movements of the steering ring 62 and thus of the swash plate 20 can be controlled via the direction of rotation of the spindle drives 22 and 24. If the spindle drives 22 and 24 are driven in the same direction of rotation, the steering ring 62 and the swash plate 20 can be pivoted about the third axis of rotation D3. If the spindle drives 22 and 24 are driven in opposite directions of rotation, the bevel toothing portions 106, 108, 110 of the bevel gears and the swash plate can be rotated about the fourth axis of rotation D4. These two movements can be superimposed in order to create complex movement patterns.

The swash plate 20 is connected via the steering wires 96 to the pivoting members or the tool tip of the tool (not shown) attached to the surgical instrument 10. In other words, the tool can be controlled via the steering wires 96, which transmit the movements generated by the steering ring 62 and the swash plate 20 to the tool. The shaft 98 extends through the swash plate 20 and the steering ring 62. The gearwheel 102, which can set the shaft S in rotation, is arranged on the shaft 98.

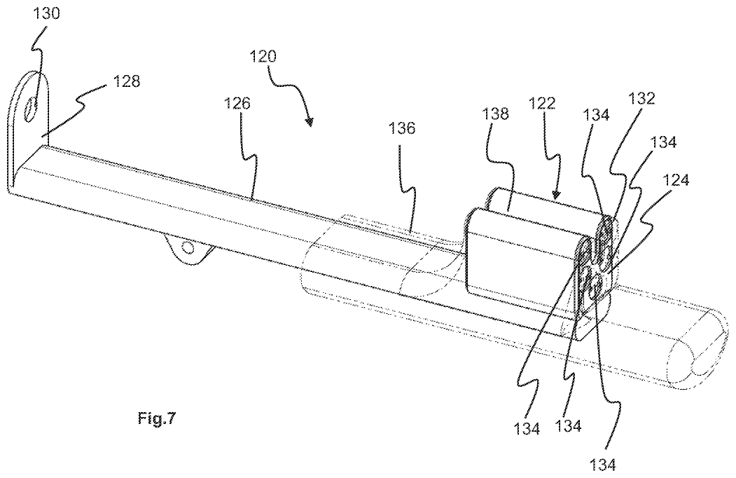

FIG. 7 shows a perspective view of a drive system 120. The drive system 120 has the drive unit 122. The drive unit 122 has an interface 124, which is designed for coupling to the surgical instrument 10. The drive unit 122 is arranged on a guide device 126, which serves for coupling to a robot arm (not shown). The guide device 126 has a guide projection 128 with a guide opening 130. A trocar (not shown) can be inserted into the guide opening 130. The shaft of a surgical instrument 10 can be guided through this guide opening 130 when the surgical instrument 10 is coupled to the drive unit 122. The sterile adapter plate 132 is arranged at the interface 124. The sterile adapter plate 132 is releasably attached to the drive unit 122 and covers the non-sterile interface 124 and, in particular, the non-sterile motor drive plates 134. The sterile adapter plate 132 has rotating clutch disks, which allow rotational movements to be transferred from the drive unit to the drive plates of the surgical instrument 10.

The sterile adapter plate 132 is connected to a sterile covering 136. The sterile covering 136 surrounds the drive unit 122 and the guide device 126 at least partially. The sterile covering 136 can be made of a film coupled to the drive unit 122. The drive unit 122 has a recess 138 formed in the shape of a groove, through which the shaft of the surgical instrument 10 can extend when the surgical instrument 10 is coupled to the drive unit 122 from the proximal direction. This means that the housing 12 of the surgical instrument 10 is located at the interface 124 of the drive unit 122, and the shaft of the surgical instrument 10 extends through the recess 138 in the form of a groove and through the guide opening 130. The recess 138 can extend in the longitudinal direction of the drive unit 122.

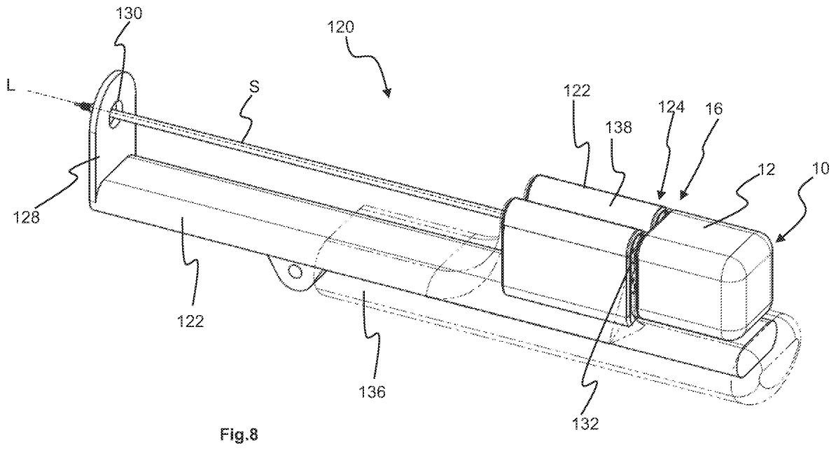

FIG. 8 shows a perspective view of the drive system 120. The surgical instrument 10 was coupled to the drive unit 122 from the proximal direction. The sterile adapter plate 132 is located between the interfaces 124 and 16 of the drive unit 122 and of the surgical instrument 10. The shaft S of the surgical instrument 10 extends through the recess 138 of the drive unit 122 and through the guide opening 130. The surgical instrument 10 can be coupled to the drive unit from proximally in the direction of the longitudinal axis L. The surgical instrument 10 or the housing 12 of the surgical instrument 10 is thus located at the end of the drive unit 122 facing away from the guide projection 128.

In FIGS. 7 and 8 it is clear that the sterile adapter plate 132 is first attached to the interface 124 of the drive unit 122. If the sterile covering 136 is connected to the sterile adapter plate 132, it can at least partially surround the drive unit 122 and the guide device 126. Following this step, the surgical instrument 10 with its housing 12, and with the steering gear 18 arranged therein, is plugged from the proximal direction onto the sterile adapter plate 132. The coupling is effected in particular in the longitudinal direction L of the surgical instrument 10. By this joining direction in a line with a trocar (not shown), the surgical instrument 10 can be pushed further distally in a continuous movement into the patient during an operation.

The present disclosure relates to a surgical instrument 10 having a housing 12 arranged at the proximal end 14 of the surgical instrument 10, an interface 16 arranged on the housing 12 for coupling to a drive unit 122, a steering gear 18 arranged in the housing 12, wherein the steering gear 18 has a spatially orientable swash plate 20, and at least one spindle drive 22, 24 for driving 22, 24 the steering gear 18. The present disclosure further relates to a sterile drive system 120 for such a surgical instrument 10 and to a method for sterile mounting of a drive system 120. The drawings, the description and the claims contain numerous features in combination. It will be appreciated that the aforementioned features can be used not only in the respectively indicated combination, but also in other combinations or in isolation, without departing from the scope of the present disclosure.

1. A surgical instrument, comprising:
a housing arranged at the proximal end of the surgical instrument,
an interface arranged on the housing for coupling to a drive unit,
a steering gear arranged in the housing, wherein the steering gear has a spatially orientable swash plate, and
at least one spindle drive for driving the steering gear.

2. The surgical instrument as set forth claim 1, wherein the interface is arranged on a coupling surface of the housing extending perpendicular to the longitudinal axis of a shaft, the shaft passing through the coupling surface.

3. The surgical instrument set forth in claim 1, wherein the surgical instrument has at least a first spindle drive and a second spindle drive, of which the axes of rotation are parallel to each other and parallel to the longitudinal axis of the surgical instrument.

4. The surgical instrument as set forth in claim 3, wherein the first spindle drive and the second spindle drive each have a drive plate arranged on the coupling surface.

5. The surgical instrument as set forth in one of claims 1 to 4, wherein the steering gear has a first slide and a second slide, and wherein the first slide is assigned to the first spindle drive and the second slide to the second spindle drive.

6. The surgical instrument as set forth in claim 5, wherein the steering gear has a clamp which is arranged on the swash plate and which is coupled to the first slide and the second slide.

7. The surgical instrument as set forth in claim 6, wherein the first slide and the second slide each have a ball head, which engage in ball sockets on the clamp.

8. The surgical instrument as set forth in claim 6, wherein the clamp has a clamp base and two clamp arms, wherein the ball sockets are arranged on end portions of the clamp arms.

9. The surgical instrument as set forth in claim 8, wherein the clamp arms define, between their end portions, an opening of the clamp, wherein the opening lies opposite the clamp base.

10. The surgical instrument as set forth in claim 6, wherein the clamp is elastic at least in some sections.

11. The surgical instrument as set forth in claim 8, wherein the elasticity of the clamp is adjustable at least via the geometric thickness and/or the material selection of the clamp base.

12. The surgical instrument as set forth in claim 6, wherein the swash plate has a groove which has mutually opposite and mutually parallel axial contact surfaces for the clamp, wherein the clamp is received in the groove.

13. The surgical instrument as set forth in claim 6, wherein the clamp surrounds more than 180°, in particular an angular range of between 270° and 350° of the circumference of the swash plate.

14. The surgical instrument as set forth in claim 1, wherein the steering gear has a steering ring coupled to the swash plate.

15. The surgical instrument as set forth in claim 14, wherein the first spindle drive and the second spindle drive interact with gearwheels for driving at least two bevel gears which are coupled to the steering ring.

16. A drive system for a surgical instrument, wherein the drive system comprises a drive unit, which can be attached to a robot arm, a sterile adapter plate, which is attached to the drive unit and at least partially covers an interface on the drive unit, and the surgical instrument as set forth in claim 1, wherein the surgical instrument is coupled to the drive unit from the proximal direction via the interface.

17. A method for sterile mounting of a drive system for a surgical instrument, wherein the drive system comprises a drive unit, which can be attached to a robot arm, and the surgical instrument as set forth in claim 1, wherein the method comprises the following steps:
attaching a sterile adapter plate to the drive unit, which at least partially covers an interface on the drive unit, and
attaching the surgical instrument to the interface of the drive unit from the proximal direction.
